(19) United States
(12) Patent Application Publication
(10) Pub. No.: US 2020/0121293 A1
(43) Pub. Date: Apr. 23, 2020

(54) MOBILE APPLICATION FOR FACILITATING SENSOR POSITIONING

(71) Applicant: Cardiac Pacemakers, Inc., St. Paul, MN (US)

(72) Inventors: Gezheng Wen, Shoreview, MN (US); Bin Mi, Arden Hills, MN (US); Stephen J. Hahn, Shoreview, MN (US); Keith R. Maile, New Brighton, MN (US)

(21) Appl. No.: 16/654,975

(22) Filed: Oct. 16, 2019

Related U.S. Application Data
(60) Provisional application No. 62/747,108, filed on Oct. 17, 2018.

(51) Int. Cl. A61B 8/08; A61B 5/00; A61B 5/0402; A61B 8/00

(52) U.S. Cl. CPC A61B 8/5269; A61B 5/684; A61B 5/7475; A61B 5/7203; A61B 8/42; A61B 5/0077; A61B 5/0402; A61B 8/5207; A61B 5/7425

(57) ABSTRACT

A medical system includes a physiological monitoring system configured to sense a physiological signal and record physiological signal data indicative of the patient's physiological state. The physiological monitoring system including a controller, a storage device, at least one sensor operatively coupled to the controller, and a first communication component. The system includes a mobile device configured to facilitate sensor placement, the mobile device comprising a controller, a display device, and a second communication component configured to facilitate communication between the physiological monitoring system and the mobile device. The controller of the mobile device is configured to provide a graphical user interface (GUI) on the display device, the GUI including information about a proper placement of the at least one sensor, wherein the proper placement is determined based on the physiological signal data.

### CROSS-REFERENCE TO RELATED APPLICATION

This application claims priority to Provisional Application No. 62/747,108, filed Sep. 17, 2018, which is herein incorporated by reference in its entirety.

### TECHNICAL FIELD

Aspects of this disclosure relate to medical devices and methods for facilitating medical monitoring. More specifically, embodiments relate to medical systems including physiological monitoring systems configured to communicate with mobile devices, and methods of using such systems.

### BACKGROUND

Wearable physiological monitoring systems may provide certain benefits. Some such systems include a number of sensors that can provide more accurate data depending upon their location with respect to each other, anatomical structures, and the like.

### SUMMARY

In an Example 1, a medical system for providing a monitoring service to a patient, the system comprising: a physiological monitoring system configured to sense a physiological signal and record physiological signal data indicative of the patient's physiological state, the physiological monitoring system including a controller, a storage device, at least one sensor operatively coupled to the controller, and a first communication component; and a mobile device configured to facilitate sensor placement, the mobile device comprising a controller, a display device, and a second communication component configured to facilitate communication between the physiological monitoring system and the mobile device; wherein the controller of the mobile device is configured to provide a graphical user interface (GUI) on the display device, the GUI including information about a proper placement of the at least one sensor, wherein the proper placement is determined based on the physiological signal data.

In an Example 2, the medical system of Example 1, wherein the controller of the physiological monitoring system is configured to determine, based on the physiological signal data, the proper placement of the at least one sensor.

In an Example 3, the medical system of Example 1, wherein the controller of the mobile device is configured to determine, based on the physiological signal data, the proper placement of the at least one sensor.

In an Example 4, the medical system of either of Examples 2 or 3, wherein the proper placement is a location determined to facilitate receiving a physiological signal having a quality that satisfies a quality criterion.

In an Example 5, the medical system of Example 4, wherein the quality criterion comprises a value of at least one of image noise, resolution, contrast, and a signal to noise ratio (SNR).

In an Example 6, the medical system of any of Examples 1-5, wherein the information about the proper placement of the at least one sensor comprises a representation of the patient's body and a representation of the at least one sensor, wherein the representation of the at least one sensor is displayed at a location relative to the representation of the patient's body that corresponds to the proper position.

In an Example 7, the medical system of any of Examples 1-4, wherein the information about the proper placement of the at least one sensor comprises instructions for positioning the at least one sensor.

In an Example 8, the medical system of any of Examples 1-6, the at least one sensor comprising a plurality of sensors, wherein the GUI provides information about a proper placement for each of the plurality of sensors.

In an Example 9, the medical system of any of Examples 1-7, wherein the physiological monitoring system comprises an acoustic imaging system, the at least one sensor comprising at least one of an acoustic transducer and an ultrasound transducer.

In an Example 10, the medical system of any of Examples 1-9, wherein the controller is further configured to receive, via the GUI, user input indicating parameters for an imaging task.

In an Example 11, the medical system of any of Examples 1-10, wherein the mobile device is configured to: receive, from the physiological monitoring system, physiological signal data; and cause the display device to present a representation of the physiological signal data.

In an Example 12, one or more computer-readable media having computer-executable instructions embodied thereon that, when executed by at least one processor, cause the at least one processor to instantiate at least one program component, the at least one program component comprising: a positioning component configured to: receive, from a physiological monitoring system, physiological signal data corresponding to a physiological signal obtained by a sensor, at a first sensor location, during a sensing task; determine, based on the physiological signal data, that a first value of a quality metric, associated with the obtained physiological signal, fails to satisfy a quality criterion; predict that a second value of the quality metric, associated with a physiological signal obtained by the sensor at a second sensor location, will satisfy the quality criterion; and provide a positioning graphical user interface (GUI) on a display device, the GUI including information to direct relocation of the sensor to the second sensor location.

In an Example 13, the media of Example 12, the at least one program component further comprising a rendering component configured to: receive, from the physiological monitoring system, physiological signal data; and cause the display device to present a representation of the physiological signal data.

In an Example 14, the media of either of Examples 12 or 13, the sensor comprising an ultrasound transducer.

In an Example 15, a method of facilitating physiological monitoring of a patient using a physiological monitoring system, the physiological monitoring system comprising a controller and at least one sensor operatively coupled to the controller, wherein the physiological monitoring system is configured to communicate with a mobile device having a display device, the method comprising: receiving, at the mobile device and from the physiological monitoring system, physiological signal data corresponding to a physiological signal obtained by a sensor, at a first sensor location, during a sensing task; determining, based on the physiological signal data, that a first value of a quality metric, associated with the obtained physiological signal sensing data, fails to satisfy a quality criterion; predicting that a second value of the quality metric, associated with a physiological signal obtained by the sensor at a second sensor location, will satisfy the quality criterion; and causing the display device to present information to direct relocation of the sensor to the second sensor location.

In an Example 16, a medical system for providing a monitoring service to a patient, the system comprising: a physiological monitoring system configured to sense a physiological signal and record physiological signal data indicative of the patient's physiological state, the physiological monitoring system including a controller, data storage circuitry, at least one sensor operatively coupled to the controller, and a first communication component; and a mobile device configured to facilitate sensor placement, the mobile device comprising a controller, a storage device, a display device, and a second communication component configured to facilitate communication between the physiological monitoring system and the mobile device, the storage device comprising one or more computer-storage media having computer-executable instructions embodied thereon that, when executed by the controller, cause the controller to instantiate at least one program component, the at least one program component comprising: a positioning component configured to provide a graphical user interface (GUI) on the display device, the GUI including information about a proper placement of the at least one sensor, wherein the proper placement is determined based on the physiological signal data.

In an Example 17, the medical system of Example 16, wherein the controller of the physiological monitoring system is configured to determine, based on the physiological signal data, the proper placement of the at least one sensor.

In an Example 18, the medical system of Example 16, wherein the controller of the mobile device is configured to determine, based on the physiological signal data, the proper placement of the at least one sensor.

In an Example 19, the medical system of Example 16, wherein the proper placement is a location determined to facilitate receiving a physiological signal having a corresponding quality metric that satisfies a quality criterion.

In an Example 20, the medical system of Example 19, wherein the quality metric comprises a value of at least one of image noise, resolution, contrast, and a signal to noise ratio (SNR).

In an Example 21, The medical system of Example 16, wherein the information about the proper placement of the at least one sensor comprises a representation of the patient's body and a representation of the at least one sensor, wherein the representation of the at least one sensor is displayed at a location relative to the representation of the patient's body that corresponds to the proper position.

In an Example 22, the medical system of Example 21, the mobile device further comprising an optical imaging component configured to obtain an image of the patient, wherein the representation of the patient's body is generated from the image of the patient.

In an Example 23, the medical system of Example 16, wherein the information about the proper placement of the at least one sensor comprises instructions for positioning the at least one sensor.

In an Example 24, the medical system of Example 16, wherein the physiological monitoring system comprises an electrocardiograph system, the at least one sensor comprising at least one electrode.

In an Example 25, the medical system of Example 16, wherein the physiological monitoring system comprises an acoustic imaging system, the at least one sensor comprising at least one ultrasound transducer.

In an Example 26, the medical system of Example 16, wherein the controller is further configured to receive, via the GUI, user input indicating parameters for an imaging task.

In an Example 27, one or more computer-readable media having computer-executable instructions embodied thereon that, when executed by at least one processor, cause the at least one processor to instantiate at least one program component, the at least one program component comprising: a positioning component configured to: receive, from a physiological monitoring system, physiological signal data corresponding to a physiological signal obtained by a sensor, at a first sensor location, during a sensing task; determine, based on the physiological signal data, that a first value of a quality metric, associated with the obtained physiological signal, fails to satisfy a quality criterion; predict that a second value of the quality metric, associated with a physiological signal obtained by the sensor at a second sensor location, will satisfy the quality criterion; and provide a positioning graphical user interface (GUI) on a display device, the GUI including information to direct relocation of the sensor to the second sensor location.

In an Example 28, the media of Example 27, the at least one program component further comprising a rendering component configured to: receive, from the physiological monitoring system, physiological signal data; and cause the display device to present a representation of the physiological signal data.

In an Example 29, the media of Example 27, the sensor comprising an ultrasound transducer.

In an Example 30, a method of facilitating physiological monitoring of a patient using a physiological monitoring system, the physiological monitoring system comprising a controller and at least one sensor operatively coupled to the controller, wherein the physiological monitoring system is configured to communicate with a mobile device having a display device, the method comprising: receiving, at the mobile device and from the physiological monitoring system, physiological signal data corresponding to a physiological signal obtained by a sensor, at a first sensor location, during a sensing task; determining, based on the physiological signal data, that a first value of a quality metric, associated with the obtained physiological signal sensing data, fails to satisfy a quality criterion; predicting that a second value of the quality metric, associated with a physiological signal obtained by the sensor at a second sensor location, will satisfy the quality criterion; and causing the display device to present information to direct relocation of the sensor to the second sensor location.

In an Example 31, the method of Example 30, wherein the physiological signal data comprises physiological signal data, and wherein determining that the first value of a quality metric fails to satisfy a quality criterion comprises: determining the first value of the quality metric; and comparing the first value of the quality metric to a quality criterion, wherein the quality criterion comprises at least one of a range and a threshold.

In an Example 32, the method of Example 31, wherein the quality metric comprises at least one of image noise, resolution, contrast, and a signal to noise ratio (SNR).

In an Example 33, the method of Example 30, wherein the physiological monitoring system comprises an acoustic imaging system, the at least one sensor comprising at least one ultrasound transducer.

In an Example 34, the method of Example 30, further comprising receiving, via a GUI, user input indicating parameters for an imaging task, and providing the parameters to the physiological monitoring system.

In an Example 35, the method of Example 30, further comprising: receiving, from the physiological monitoring system, physiological signal data; and causing the display device to present a representation of the physiological signal data.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of a medical system including a physiological monitoring system and a mobile device, in accordance with embodiments of the disclosure.

FIG. 2 is a block diagram of a computing device, in accordance with embodiments of the disclosure.

FIG. 3 is a block diagram of a medical system having a physiological monitoring system and a mobile device, in accordance with embodiments of the disclosure.

FIG. 4 is an illustrative graphical user interface (GUI) depicting representations of sensor locations on a representation of a patient, in accordance with embodiments of the disclosure.

FIG. 5 is a flow diagram illustrating a method of facilitating physiological monitoring, in accordance with embodiments of the disclosure.

While the disclosed subject matter is amenable to various modifications and alternative forms, specific embodiments have been shown by way of example in the drawings and are described in detail below. The intention, however, is not to limit the subject matter disclosed herein to the particular embodiments described. On the contrary, the disclosure is intended to cover all modifications, equivalents, and alternatives falling within the scope of the subject matter disclosed herein, and as defined by the appended claims.

As used herein in association with values (e.g., terms of magnitude, measurement, and/or other degrees of qualitative and/or quantitative observations that are used herein with respect to characteristics (e.g., dimensions, measurements, attributes, components, etc.) and/or ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a value, configuration, orientation, and/or other characteristic that is equal to (or the same as) the stated value, configuration, orientation, and/or other characteristic or equal to (or the same as) a value, configuration, orientation, and/or other characteristic that is reasonably close to the stated value, configuration, orientation, and/or other characteristic, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

The terms “up,” “upper,” and “upward,” and variations thereof, are used throughout this disclosure for the sole purpose of clarity of description and are only intended to refer to a relative direction (i.e., a certain direction that is to be distinguished from another direction), and are not meant to be interpreted to mean an absolute direction. Similarly, the terms “down,” “lower,” and “downward,” and variations thereof, are used throughout this disclosure for the sole purpose of clarity of description and are only intended to refer to a relative direction that is at least approximately opposite a direction referred to by one or more of the terms “up,” “upper,” and “upward,” and variations thereof.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

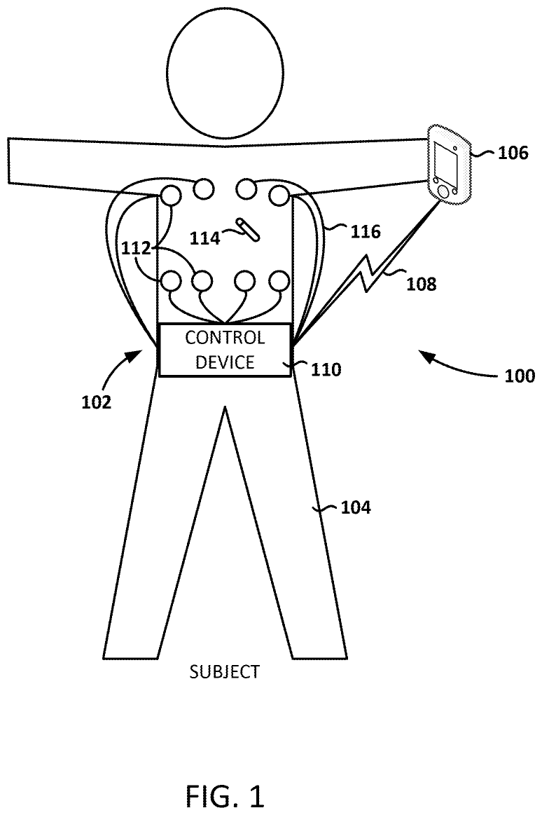

FIG. 1 shows an illustrative medical system 100, in accordance with embodiments of the disclosure. As shown in FIG. 1, the medical system 100 includes a physiological monitoring system 102 configured to be operatively connected to the body of a subject 104, and a mobile device 106, which is communicatively coupled to the monitoring system 102 via a communication link 108. In the illustrated embodiments, the medical system 100 is operatively coupled to the subject 104, and the monitoring system 102 is configured to communicate with the mobile device 106 over the communication link 108. The subject 104 may be a human, a dog, a pig, and/or any other animal having physiological parameters that can be recorded. For example, in embodiments, the subject 104 may be a human patient.

In embodiments, the communication link 108 may be, or include, a wireless communication link such as, for example, a short-range radio link, such as Bluetooth, IEEE 802.11, a proprietary wireless protocol, and/or the like. In embodiments, for example, the communication link 108 may utilize Bluetooth Low Energy radio (Bluetooth 4.1), or a similar protocol, and may utilize an operating frequency in the range of 2.40 to 2.48 GHz. The term “communication link” may refer to an ability to communicate some type of information in at least one direction between at least two devices, and should not be understood to be limited to a direct, persistent, or otherwise limited communication channel. That is, according to embodiments, the communication link 108 may be a persistent communication link, an intermittent communication link, an ad-hoc communication link, and/or the like. The communication link 108 may refer to direct communications between the monitoring system 102 and the mobile device 106, and/or indirect communications that travel between the monitoring system 102 and the mobile device 106 via at least one other device (e.g., a repeater, router, hub, and/or the like). The communication link 108 may facilitate uni-directional and/or bi-directional communication between the monitoring system 102 and the mobile device 106. Data and/or control signals may be transmitted between the monitoring system 102 and the mobile device 106 to coordinate the functions of the monitoring system 102 and the mobile device 106. In embodiments, patient data may be downloaded from one or more of the monitoring system 102 and the mobile device 106 periodically or on command. The physician and/or the patient may communicate with the monitoring system 102 and the mobile device 106, for example, to acquire patient data or to initiate, terminate and/or modify recording and/or therapy.

In embodiments, the monitoring system 102 and/or the mobile device 106 may provide one or more of the following functions with respect to a patient: sensing, data analysis, and therapy. For example, in embodiments, the monitoring system 102 and/or the mobile device 106 may be used to measure any number of a variety of physiological, device, subjective, and/or environmental parameter signals associated with the subject 104, using electrical, mechanical, and/or chemical means. The monitoring system 102 and/or the mobile device 106 may be configured to automatically gather data, gather data upon request (e.g., input provided by the subject, a clinician, another device, and/or the like), and/or any number of various combinations and/or modifications thereof. The monitoring system 102 and/or the mobile device 106 may be configured to compress and/or store data related to the physiological, device, environmental, and/or subjective parameter signals and/or transmit the data to any number of other devices in the system 100. In embodiments, the monitoring system 102 and/or the mobile device 106 may be configured to analyze data and/or act upon the analyzed data. For example, the monitoring system 102 and/or the mobile device 106 may be configured to modify therapy, perform additional monitoring, and/or provide alarm indications based on the analysis of the data.

According to embodiments, the monitoring system 102 may include any number of different types of medical devices, any number of different components of an implantable and/or external system, and/or the like. For example, the monitoring system 102 may include a control device 110 configured to communicate with and/or control one or more sensors 112, one or more monitoring devices 114, a pacemaker, an implantable cardioverter defibrillator (ICD), a cardiac resynchronization therapy (CRT) device and/or the like. According to embodiments, the control device 110 may be integrated with one or more of the sensors 112, monitoring devices 114, and/or the like. In embodiments, the control device 110 may be a separate device and may be worn by the user, carried by the user, placed adjacent the user, and/or the like.

In embodiments, the one or more sensors 112 may include any number of different types of sensors configured to obtain physiological signals (e.g., data associated with the subject's body and its processes), environmental signals (e.g., temperature, humidity, pressure, acceleration, etc.), position signals (e.g., GPS) and/or the like. For example, in embodiments, a sensor 112 may be an electrode, an acoustic transducer, an optical sensor, an accelerometer, a barometer, a thermometer, and/or the like. According to embodiments, any one or more of the sensors 112 may be communicatively coupled to the control device 110 via a wire, a wireless link, and/or the like. In embodiments, for example, the physiological monitoring system 102 may be an electrocardiograph system and one or more of the sensors 112 may be an electrode. In embodiments, the physiological monitoring system may be an acoustic imaging system, and one or more of the sensors 112 may be an acoustic transducer.

In embodiments, a monitoring device 114 may include an external device and/or an internal device. For example, in embodiments, the monitoring system 102 may include one or more implantable medical devices 114 (IMDs) implanted subcutaneously within an implantation location or pocket in the patient's chest or abdomen and may be configured to monitor (e.g., sense and/or record) physiological parameters associated with the patient's heart. In embodiments, the IMD 114 may be an implantable cardiac monitor (ICM) (e.g., an implantable diagnostic monitor (IDM), an implantable loop recorder (ILR)) configured to record physiological parameters such as, for example, one or more cardiac electrical signals, heart sounds, heart rate, blood pressure measurements, oxygen saturations, and/or the like.

In embodiments, the physiological monitoring system 102 may include sensing components such as, for example, one or more surface electrodes configured to obtain an electrocardiogram (ECG), one or more accelerometers and/or gyroscopes configured to detect motion associated with the subject 104, one or more respiratory sensors configured to obtain respiration information, one or more environmental sensors configured to obtain information about the external environment (e.g., temperature, air quality, humidity, carbon monoxide level, oxygen level, barometric pressure, light intensity, sound, and/or the like) surrounding the subject 104, and/or the like. In embodiments, the physiological monitoring system 102 may be configured to measure parameters relating to the human body, such as temperature (e.g., a thermometer), blood pressure (e.g., a sphygmomanometer), blood characteristics (e.g., glucose levels), body weight, physical strength, mental acuity, diet, heart characteristics, relative geographic position (e.g., a Global Positioning System (GPS)), and/or the like.

According to embodiments, the physiological monitoring system 102 and/or the mobile device 106 may be configured to measure subjective and/or perceptive data from the subject 104. Subjective data is information related to a patient's feelings, perceptions, and/or opinions, as opposed, for example, to objective physiological signal data. For example, physiological monitoring system 102 and/or the mobile device 106 may be configured to measure subject responses to inquiries such as “How do you feel?” and “How is your pain?” The physiological monitoring system 102 and/or the mobile device 106 may be configured to prompt the subject 104 and record subjective data from the subject 104 using visual and/or audible cues. In embodiments, the subject 104 can press coded response buttons or type an appropriate response on a keypad or provide a response using a graphical user interface provided by the mobile device 106. In embodiments, subjective data may be collected by allowing the subject 104 to speak into a microphone and using speech recognition software to process the subjective data.

In embodiments, the physiological monitoring system 102 and/or the mobile device 106 may be configured to monitor physiological parameters that may include one or more signals indicative of a patient's physical activity level and/or metabolic level, such as an acceleration signal. In embodiments, the physiological monitoring system 102 and/or the mobile device 106 may be configured to sense intrathoracic impedance, from which various respiratory parameters may be derived, including, for example, respiratory rate, tidal volume and minute ventilation. Sensors and associated circuitry may be incorporated in connection with the physiological monitoring system 102 and/or the mobile device 106 for detecting one or more body movement or body posture and/or position related signals. For example, accelerometers, gyroscopes, and/or GPS devices may be employed to detect patient activity, patient location, body orientation, and/or torso position. The physiological monitoring system 102 and/or the mobile device 106 may be configured to sense and/or record at regular intervals, continuously, and/or in response to a detected event.

In various embodiments, the physiological monitoring system 102 may be a system that is configured to be portable with the subject 104, e.g., by being integrated into a vest, belt, harness, sticker; placed into a pocket, a purse, or a backpack; carried in the subject's hand; integrated with a mobile patient bed; and/or the like, or otherwise operatively (and/or physically) coupled to the subject 104 (e.g., integrated into an operating room table/bed). In embodiments, the physiological monitoring system 102 may be, or include, a wearable cardiac defibrillator (WCD) such as a vest that includes one or more defibrillation electrodes. In embodiments, the physiological monitoring system 102 may be, or include, a wearable garment that includes one or more acoustic transducers, ultrasound transducers, and/or the like. In embodiments, the physiological monitoring system 102 may be, or include, a garment (e.g., configured to be worn under or over clothes) that includes a number of different pockets into which sensors 112 may be placed. In this manner, for example, one or more sensors 112 may be relocated from a first sensor location to a second sensor location (e.g., to a different pocket) such as, for example, in response to an indication to do so being presented by the mobile device 106 (e.g., to improve signal quality). According to embodiments, the sensors 112 may be operatively coupled to the body of the subject 104 in other manners such as, for example, using adhesive, straps, and/or the like. In embodiments, sensors 112 may be disposed in a bed or chair on which the patient lays or sits such as, for example, by integrating the sensors 112 into pockets built into a mattress, a pad that can be placed on top of a mattress, and/or the like.

In embodiments, the physiological monitoring system 102 may include any number of different therapy components such as, for example, a defibrillation component, a drug delivery component, a neurostimulation component, a neuromodulation component, a temperature regulation component, and/or the like. In embodiments, the physiological monitoring system 102 may include limited functionality, e.g., defibrillation shock delivery and communication capabilities, with arrhythmia detection, classification and/or therapy command/control being performed by a separate device such as, for example, the IMD 114 and/or the mobile device 106.

Although not shown in FIG. 1, the system 100 may include any number of external communication devices, programmers, servers, and/or the like. According to embodiments, an external communication device and/or a programmer may be configured to send data to, and receive data from, a device, such as the control device 110, the IMD 114, the mobile device 106, and/or any number of other devices depicted or not depicted in FIG. 1. Such communications may be facilitated via any number of communication links, any number of which may be, be identical to, be similar to, include, be coupled with, or be included within, the communication link 108.

The illustrative medical system 100 shown in FIG. 1 is not intended to suggest any limitation as to the scope of use or functionality of embodiments of the present disclosure. The illustrative system 100 also should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the present disclosure.

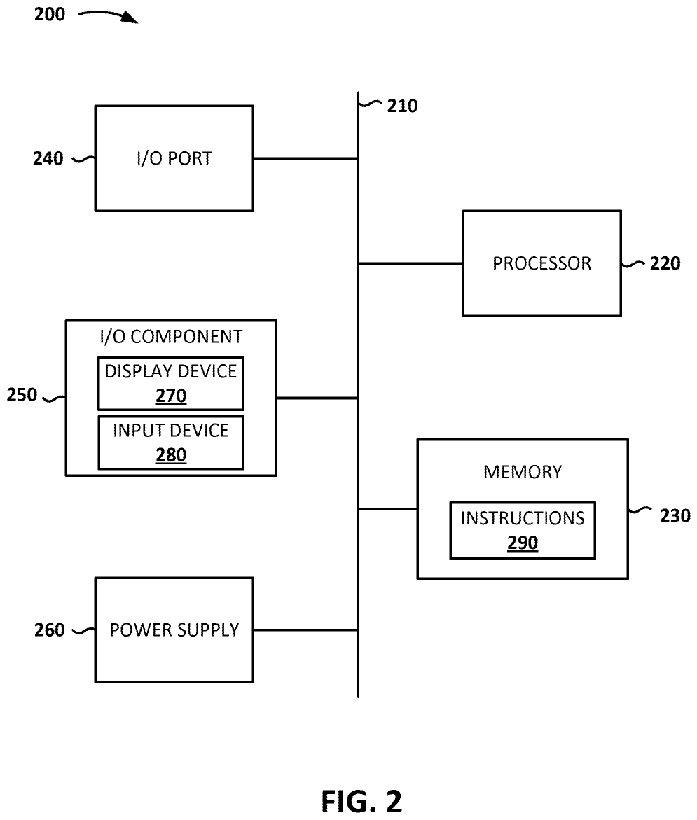

According to various embodiments of the disclosed subject matter, any number of the components depicted in FIG. 1 (e.g., the mobile device 106, the control device 110, etc.) may be implemented on one or more computing devices. FIG. 2 is a block diagram depicting an illustrative computing device 200, in accordance with embodiments of the disclosure. The computing device 200 may include any type of computing device suitable for implementing aspects of embodiments of the disclosed subject matter. Examples of computing devices include specialized computing devices or general-purpose computing devices such “workstations,” “servers,” “laptops,” “desktops,” “tablet computers,” “hand-held devices,” “general-purpose graphics processing units (GPGPUs),” and the like, all of which are contemplated within the scope of FIGS. 1 and 2, with reference to various components of the system 100 and/or computing device 200.

In embodiments, the computing device 200 includes a bus 210 that, directly and/or indirectly, couples the following devices: a processor 220, a memory 230, an input/output (I/O) port 240, an I/O component 250, and a power supply 260. Any number of additional components, different components, and/or combinations of components may also be included in the computing device 200. The I/O component 250 may include a presentation component configured to present information to a user such as, for example, a display device 270, a speaker, a printing device, and/or the like, and/or an input device 280 such as, for example, a microphone, a joystick, a satellite dish, a scanner, a printer, a wireless device, a keyboard, a pen, a voice input device, a touch input device, a touch-screen device, an interactive display device, a mouse, and/or the like.

The bus 210 represents what may be one or more busses (such as, for example, an address bus, data bus, or combination thereof). Similarly, in embodiments, the computing device 200 may include a number of processors 220, a number of memory components 230, a number of I/O ports 240, a number of I/O components 250, and/or a number of power supplies 260. Additionally any number of these components, or combinations thereof, may be distributed and/or duplicated across a number of computing devices.

In embodiments, the memory 230 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory 230 stores computer-executable instructions 290 for causing the processor 220 to implement aspects of embodiments of system components discussed herein and/or to perform aspects of embodiments of methods and procedures discussed herein.

The computer-executable instructions 290 may include, for example, computer code, machine-useable instructions, and the like such as, for example, program components capable of being executed by one or more processors 220 associated with the computing device 200. Program components may be programmed using any number of different programming environments, including various languages, development kits, frameworks, and/or the like. Some or all of the functionality contemplated herein may also, or alternatively, be implemented in hardware and/or firmware.

The illustrative computing device 200 shown in FIG. 2 is not intended to suggest any limitation as to the scope of use or functionality of embodiments of the present disclosure. The illustrative computing device 200 also should not be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the present disclosure.

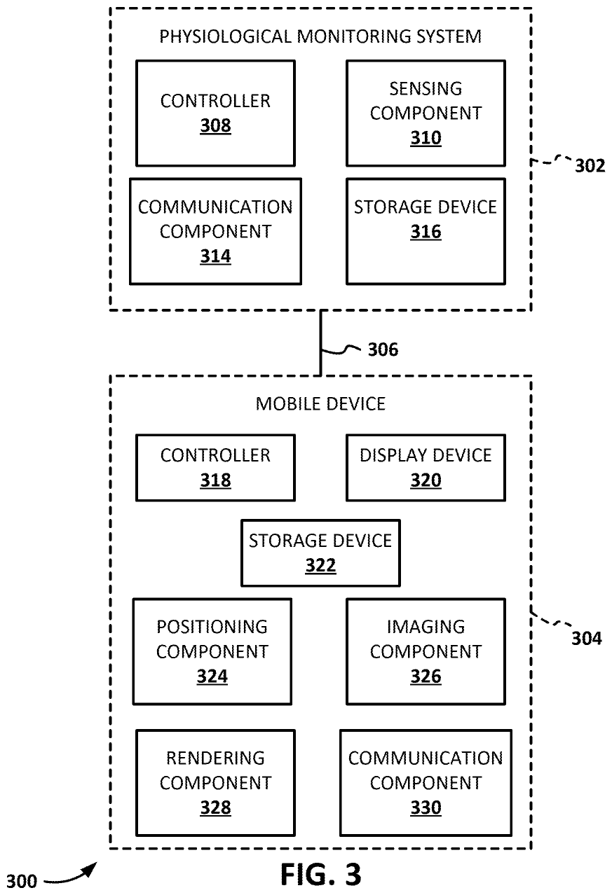

FIG. 3 is a block diagram depicting an illustrative medical system 300, in accordance with embodiments of the disclosure. As shown, the system 300 includes a physiological monitoring system 302 and a mobile device 304 configured to communicate with one another via a communication link 306. Embodiments of the system may include more than one physiological monitoring system 302 and/or more than one mobile device 304. The physiological monitoring system 302 may be, be similar, to, include, or be included in, the physiological monitoring system 102 depicted in FIG. 1; and the mobile device 304 may be, be similar to, include, or be included in, the mobile device 106 depicted in FIG. 1.

According to embodiments illustrated in FIG. 3, the physiological monitoring system 302 includes a controller 308, a sensing component 310, a communication component 314, and a storage device 316. The controller 308 may include, for example, a processing unit, a pulse generator, and/or the like. The controller 308 may be any arrangement of electronic circuits, electronic components, processors, program components and/or the like configured to store and/or execute programming instructions, to direct the operation of the other functional components of the physiological monitoring system 302, to perform arrhythmia detection and/or classification algorithms, to perform ultrasound imaging tasks (e.g., identification of B-lines for diagnosing and/or confirming pulmonary edema), to store physiologic data obtained by the sensing component 310, and/or the like, and may be implemented, for example, in the form of any combination of hardware, software, and/or firmware.

According to embodiments, for example, the physiological monitoring system 302 may include, as part of the sensing component 310, an array of ultrasound transducers configured to obtain ultrasound imaging signals of an interior region of a patient's body, which the controller 308 may process to produce ultrasound images, detect and/or characterize B-lines, and/or the like. In embodiments, for example, the system 302 may include a number of ultrasound sensors configured to be placed on the patient. The ultrasound sensors may be retained in a wearable garment, positioned on, in, or under a patient table, and/or the like. According to embodiments, the controller 308 may be configured to analyze ultrasound images obtained using the ultrasound transducers to detect the presence of B-lines, the prevalence of B-lines, and/or otherwise characterize B-lines in the ultrasound images. In embodiments, the controller 308 may be configured to analyze and/or provide representations of trends of B-line measurements, use B-line measurements to track for pulmonary edema, pulmonary hypertension, early warning of congestion decompensation, and/or the like.

Additionally or alternatively, the sensing component 310 may include an number of ultrasound transducers and/or acoustic sensors configured to obtain acoustic signals that the controller 308 may be configured to analyze to determine any number of different parameters such as, for example, based on attenuation of the acoustic signals as they travel through the patient's body. It should be understood that the above-described ultrasound imaging, acoustic imaging, and/or the like may be performed by any number of components configured to operate as an acoustic imaging system, and that ultrasound sensors are a specific type of acoustic sensor. Thus, as the term is used herein, an acoustic sensor may include any number of different types of acoustic sensors, including ultrasound sensors, and/or the like.

In embodiments, the controller 308 may be a programmable micro-controller or microprocessor, and may include one or more programmable logic devices (PLDs) or application specific integrated circuits (ASICs). In some implementations, the controller 308 may include memory as well. Although embodiments of the present system 300 are described in conjunction with a physiological monitoring system 302 having a processor-based architecture, it will be understood that the physiological monitoring system 302 (or other device) may be implemented in any logic-based integrated circuit architecture, if desired. The controller 308 may include digital-to-analog (D/A) converters, analog-to-digital (ND) converters, timers, counters, filters, switches, and/or the like. The controller 308 may execute instructions and perform desired tasks as specified by the instructions.

The controller 308 may also be configured to store information in the storage device 316 and/or access information from the storage device 316. The storage device 316 may be, be similar to, include, or be included within, the memory 230 depicted in FIG. 2. That is, for example, the storage device 316 may include volatile and/or non-volatile memory, and may store instructions that, when executed by the controller 308 cause methods and processes to be performed by the physiological monitoring system 302. In embodiments, the controller 308 may process instructions and/or data stored in the physiological monitoring system 302 to control sensing operations performed by the physiological monitoring system 302, to control communications performed by the physiological monitoring system 302, and/or the like. For example, the controller 308 may include a processing component configured to process a sensed signal, e.g., to determine parameters, derived features, and/or the like.

The physiological monitoring system 302 may sense physiological parameter signals using a sensing component 310 that may include, for example, one or more sensors (e.g., the one or more sensors 112 depicted in FIG. 1), one or more medical devices (e.g., the one or more medical devices 114 depicted in FIG. 1), or a combination of these. In embodiments, the sensing component 310 may include any number of electrical circuits, electronic components, processors, program components and/or the like. The storage device 316 may be used to store sensed information according to some implementations. Information from sense circuits included in the sensing component 310 may be used to adjust therapy, sensing, and/or communications parameters.

In embodiments, the sensing component 310 may be configured to sense intrinsic cardiac electrical signals in a manner similar to known electrocardiogram (ECG) electrodes, which signals are transmitted via conventional techniques to the controller 308. In various embodiments, the sensing component 310 may be configured to sense other patient physiologic or environmental parameters in addition to, or alternative to, cardiac signals. In embodiments, the sensing component 310 may include temperature sensors (e.g., thermocouples or thermistors), barometers, acoustic transducers, pressure sensors, optical sensors, motion or impact sensors (e.g., accelerometers, inertial measuring units (IMUs)), strain sensors, Doppler systems, ultrasound sensors, and/or the like, in any number of various types of configurations. The foregoing sensors allow the physiological monitoring system 302 to be capable of sensing and recording physiologic parameters such as, for example, patient movement, posture, respiratory rate and/or volume, heart sounds, impedance, fluid levels, and/or the like. The output from the sensing component 310 may be used in arrhythmia detection and classification, pulmonary edema diagnosis and/or confirmation, therapy selection, and/or the like.

The communication component 314 may include, for example, circuits, program components, and one or more transmitters and/or receivers for communicating wirelessly with one or more other devices such as, for example, the mobile device 304. According to various embodiments, the communication component 314 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 314 may include any combination of hardware, software, and/or firmware configured to facilitate establishing, maintaining, and using any number of communication links.

As shown in FIG. 3, the mobile device 304 includes a controller 318, a display device 320, a storage device 322, a positioning component 324, an optical imaging component 326, a rendering component 328, and a communication component 330. In embodiments, the positioning component 324, optical imaging component 326, rendering component 328, and/or communication component 330 may be implemented in any combination of hardware, software, and/or firmware, and may be implemented, at least in part, by the controller 318. The controller 318 may be any suitable controller 308 configured for implementation in a mobile device and may be identical to, or similar to, the controller 308 of the physiological monitoring system 302.

Additionally, the storage device 322 and communication component 330 may be identical to, or similar to, the storage device 310 and the communication component 316, respectively, of the physiological monitoring system 302. That is, for example, the storage device 322 may include volatile and/or non-volatile memory, and may store instructions that, when executed by the controller 318 cause methods and processes to be performed by the mobile device 304. In embodiments, the controller 318 may process instructions and/or data stored in the storage device 322 to control communications performed by the communication component 330. The mobile device 304 may include any number of other components or combination of components including, for example, a sensing component, a therapy component, and/or the like.

According to embodiments, the positioning component 324 may be configured to facilitate positioning the one or more sensors adjacent (or against) the body of the patient to facilitate obtaining physiological signal data of a certain quality. In embodiments, the positioning component 324 may be configured to provide a graphical user interface (GUI) on the display device 320, the GUI including information about a proper placement of a sensor (or sensors), where the proper placement is determined based on physiological signal data obtained by the sensor. According to embodiments, the positioning component 324 may be configured to determine a proper placement by determining a sensor location such that a physiological signal received by a sensor at that sensor location is predicted to have a corresponding quality metric that satisfies a quality criterion.

According to embodiments, the positioning component 324 may be configured to determine a first sensor location of a sensor in any number of ways. For example, in embodiments, the positioning component 324 may receive information from the physiological monitoring system 302 indicating the first sensor location. In embodiments, the physiological monitoring system 302 and/or the mobile device 304 may be configured to register the position of a sensor with respect to anatomical features identified by the system 300 based on physiological signal data obtained by the sensor. In embodiments, an approximate sensor location may be determined based on a known placement of the sensor with respect to other sensors. That is, for example, where the physiological monitoring system 302 includes a garment, pad, or other structure configured to retain the sensors, information may be stored in one or more of the storage devices 316 or 322 that indicates sensor positions on the structure. The information may include information about the relative location of each sensor position to each other sensor position. That relative location can be analyzed, by the positioning component 324, in conjunction with any available information about the positioning of the structure on the body of the patient to determine an approximate sensor location. Embodiments may include other ways of determining an approximate sensor location. For example, the optical imaging component 326 may be used to obtain an image of the patient and that image may be analyzed to identify sensors and their approximate locations.

The approximate sensor location may be refined and/or confirmed based on physiological signal data obtained by sensor and/or one or more other sensors. That is, for example, electrodes may be used to determine impedance measurements that can facilitate identifying anatomical structures adjacent the sensor. Acoustic sensors may be used to identify anatomical structures adjacent the sensor by analyzing acoustic signal attenuation, performing ultrasound imaging, and/or the like. Any number of other types of sensors may be used to facilitate determining a sensor location corresponding to a sensor. In embodiments, machine learning techniques may be used to improve the positioning component's 324 ability to determine sensor locations over time. In embodiments, for example, a number of different types of information may be used with deep learning to train models that can be used by the positioning component 324 to determine sensor locations. According to embodiments, any number of different aspects of these processes may be performed additionally, or alternatively, by the physiological monitoring system 302 and/or one or more servers (not shown).

To determine whether a sensor's current location is appropriate, the positioning component 324 may be configured to determine whether a quality metric associated with physiological signal data corresponding thereto satisfies a quality criterion or criteria. In embodiments, for example, the quality metric may be, or include, a value of at least one of image noise, resolution, contrast, a signal to noise ratio (SNR), and/or the like. The positioning component 324 may be configured to receive, from the physiological monitoring system 302, physiological signal data corresponding to a physiological signal obtained by a sensor, at a first sensor location, during a sensing task and to determine, based on the physiological signal data, a first value of the quality metric. According to embodiments, physiological signal data may include raw signal data sensed by a physiological sensor and/or information derived therefrom.

The positioning component 324 may be further configure to compare the first value of the quality metric to one or more quality criteria to determine whether the first value of the quality metric satisfies the one or more quality criteria. According to embodiments, for example, a quality criterion may include a specified threshold and/or a range of values. According to embodiments, any number of different quality metrics may be used. Additionally, machine learning techniques may be used to improve the positioning component's 324 ability to determine quality metric values, quality criteria, whether quality metric values satisfy one or more quality criteria, and/or the like.

Upon determining that a quality metric value fails to satisfy a quality criterion (or criteria), the positioning component 324 may be configured to predict that a second value of the quality metric, associated with a physiological signal obtained by the sensor at a second sensor location, will satisfy the quality criterion. According to embodiments, any number of different machine-learning techniques may be used to facilitate this prediction. For example, the positioning component 324 may use classifiers, neural networks, deep learning, and/or the like, to analyze physiological signal data obtained by any number of sensors to make this prediction. According to embodiments, the positioning component 324 may be configured to determine a second sensor location at which the signal quality is likely to be highest.

In embodiments in which the physiological monitoring system includes a structure for retaining the sensors in predetermined sensor positions (relative to the structure and each other), the positioning component 324 may be configured to analyze predicted signal quality metrics for any number of sensor locations different than the first sensor location. In embodiments, for example, the positioning component 324 may be configured to first determine a predicted signal quality metric for the sensor location nearest the first sensor location, and if that sensor location is predicted to yield physiological signal data that has a corresponding signal quality metric that satisfies the quality criterion, the positioning component 324 may identify that sensor location as the second (“appropriate”) sensor location; and, if not, the positioning component 324 may be configured to perform the same analysis with respect to the next closest sensor location. According to embodiments, the positioning component 324 may utilize any number of other processes to facilitate determining a second (more appropriate”) sensor location.

According to embodiments, the positioning component 324 may be further configured to provide, via a GUI, information about the second sensor location (e.g., “proper placement of the sensor”). In embodiments, the information about the proper placement of a sensor may include a representation of the patient's body and a representation of the sensor, where the representation of the sensor is displayed at a location relative to the representation of the patient's body that corresponds to the determined second (“proper”) position. According to embodiments, for example, the positioning component may be configured to receive, from the imaging component 326, an image of the patient and to render a representation of that image on the display device 320. The representation of the image may be the image itself or an image derived therefrom. A representation of the sensor may be displayed on the representation of the patient's body to demonstrate the determined proper placement of the sensor.

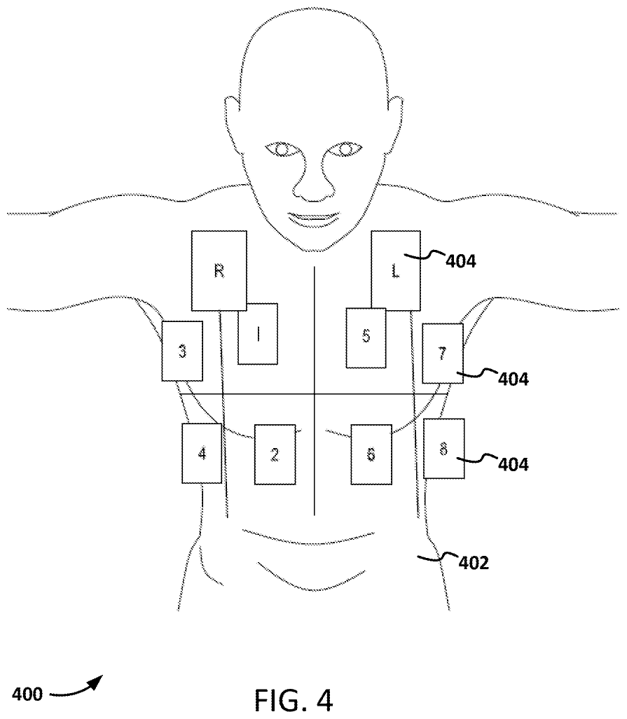

For example, turning briefly to FIG. 4, an illustrative GUI 400 is depicted, in which a representation 402 of a patient's body is presented. A number of representations 404 of sensor locations are superimposed on the representation 402 of the patient's body to inform a user of the locations at which certain sensors should be placed. In embodiments, as shown, the positioning component 324 may be configured to present representations of a number of sensor locations, each corresponding to a different sensor. As shown, for example, each sensor location may be labeled with a number or letter that corresponds to a particular sensor. To facilitate proper placement, each sensor may also be labeled with the corresponding number or letter. In embodiments, anatomical landmarks can be displayed with notations and/or markings and the approximate distance between the anatomical landmark and a sensor location. For example, in embodiments, a marking (e.g., a red dot, a symbol, etc.) may be used to indicate a sternum notch.

The illustrative GUI 400 shown in FIG. 4 is not intended to suggest any limitation as to the scope of use or functionality of embodiments of the present disclosure. The illustrative GUI 400 also should not be interpreted as having any dependency or requirement related to any single component or combination of components illustrated therein. Additionally, various components depicted in FIG. 4 may be, in embodiments, integrated with various ones of the other components depicted therein (and/or components not illustrated), all of which are considered to be within the ambit of the present disclosure.

With renewed focus on FIG. 3, the positioning component 324 may, additionally or alternatively, be configured to provide instructions to a user for positioning the at least one sensor. The instructions may include written text, audio, and/or video presentations. In embodiments, the positioning component 324 may be configured to provide real-time feedback regarding the relocation of the sensor. That is, for example, the positioning component 324 may provide, via the GUI, an indication of the relative position of the sensor with respect to the determined second location. The indication may include a graphic in which the color changes to indicate that the sensor is getting closer to the second sensor location, sound feedback, and/or the like. Upon determining that the sensor has been placed in the determined second sensor location, the positioning component 324 may be configured to provide an indication, via the GUI, that the relocation of the sensor has been successful. In embodiments, the positioning component 324 may be configured to determine whether physiological signal data received by the sensor at the second sensor location includes quality metrics that satisfy the quality criterion before indicating a successful relocation of the sensor.

The optical imaging component 326 may be configured to obtain images, and may include all of the technology that works together to perform that function. For example, the optical imaging component 326 may include an optical imaging device (e.g., a camera), software and/or firmware that facilitates controlling the imaging device and processing image data obtained therefrom. According to embodiments, the optical imaging component 326 may be used, for example, to obtain an image of the patient, where the representation of the patient's body is generated from the image of the patient.

The rendering component 328 may be configured to receive, from the physiological monitoring system, physiological signal data; and cause the display device to present a representation of the physiological signal data. According to embodiments, the rendering component 328 may be configured to interpret, analyze, and/or otherwise process physiological signal data prior to presenting representations thereof. In embodiments, the rendering component 328 may provide, via a GUI, interactive representations of physiological signal data. Representations of physiological signal data may include, for example, parameter values, indications of diagnoses, graphs, charts, anatomical maps, images (e.g., ultrasound images), and/or the like. According to embodiments, the rendering component 328 may also be configured to receive, via a GUI, inputs from a user that indicate parameter settings for a particular sensing task. That is, for example, the GUI may facilitate user control of any number of aspects of operation of the physiological monitoring system 302.

The illustrative medical system 300 shown in FIG. 3 is not intended to suggest any limitation as to the scope of use or functionality of embodiments of the present disclosure. The illustrative medical system 300 also should not be interpreted as having any dependency or requirement related to any single component or combination of components illustrated therein. Additionally, various components depicted in FIG. 3 may be, in embodiments, integrated with various ones of the other components depicted therein (and/or components not illustrated), all of which are considered to be within the ambit of the present disclosure.

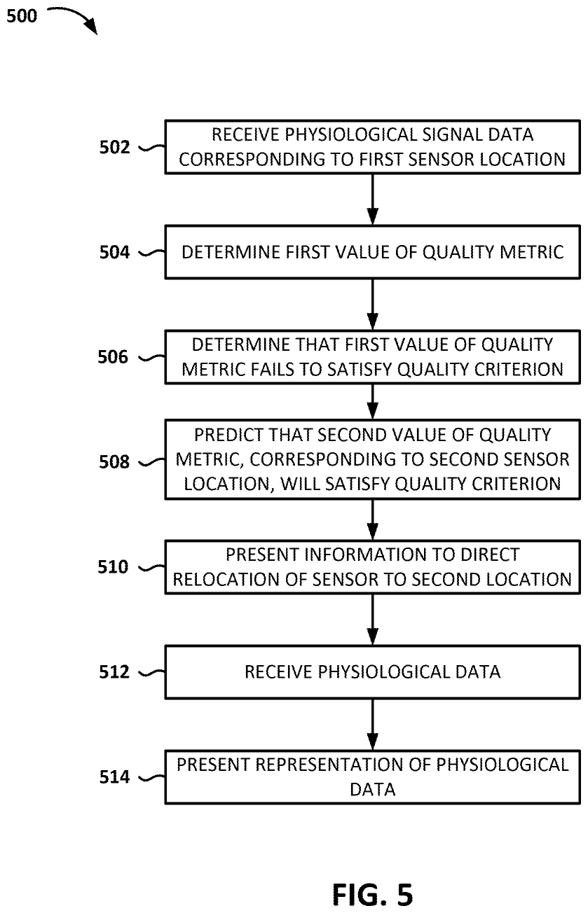

FIG. 5 is a flow diagram depicting an illustrative method 500 of facilitating physiological monitoring of a patient, using a physiological monitoring system in accordance with embodiments of the disclosure. The method may be performed, for example, by a mobile device having a display device (e.g., the mobile device 106 depicted in FIG. 1 and/or the mobile device 304 depicted in FIG. 3), a server, and/or the like. According to embodiments, the physiological monitoring system may be, be similar to, include, or be included in, the physiological monitoring system 102 depicted in FIG. 1, and/or the physiological monitoring system 302 depicted in FIG. 3. That is, for example, the physiological monitoring system may include a controller and at least one sensor operatively coupled to the controller, where the physiological monitoring system is configured to communicate with the mobile device.

Embodiments of the method 500 may be performed by aspects of a medical system (e.g., the physiological monitoring system 102 depicted in FIG. 1, the mobile device 106 depicted in FIG. 1, the physiological monitoring system 302 depicted in FIG. 3, the mobile device 304 depicted in FIG. 3, etc.). Embodiments of the method 500 include receiving, at the mobile device and from the physiological monitoring system, physiological signal data corresponding to a physiological signal obtained by a sensor, at a first sensor location, during a sensing task (block 502). As shown in FIG. 5, embodiments of the method 500 further include determining the first value of the quality metric (block 504) and determining, based on the physiological signal data, that a first value of a quality metric, associated with the obtained physiological signal sensing data, fails to satisfy a quality criterion (block 506). For example, this determination may be made by comparing the first value of the quality metric to a quality criterion, wherein the quality criterion includes at least one of a range and a threshold.

Embodiments of the method 500 further include predicting that a second value of the quality metric, associated with a physiological signal obtained by the sensor at a second sensor location, will satisfy the quality criterion (block 508); and causing the display device to present information to direct relocation of the sensor to the second sensor location (block 510). In embodiments, the method 500 also may include receiving, from the physiological monitoring system, physiological signal data (block 512) and causing the display device to present a representation of the physiological signal data (block 514).

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical system for providing a monitoring service to a patient, the system comprising:
a physiological monitoring system configured to sense a physiological signal and record physiological signal data indicative of the patient's physiological state, the physiological monitoring system including a controller, data storage circuitry, at least one sensor operatively coupled to the controller, and a first communication component; and
a mobile device configured to facilitate sensor placement, the mobile device comprising a controller, a storage device, a display device, and a second communication component configured to facilitate communication between the physiological monitoring system and the mobile device, the storage device comprising one or more computer-storage media having computer-executable instructions embodied thereon that, when executed by the controller, cause the controller to instantiate at least one program component, the at least one program component comprising:
a positioning component configured to provide a graphical user interface (GUI) on the display device, the GUI including information about a proper placement of the at least one sensor, wherein the proper placement is determined based on the physiological signal data.

2. The medical system of claim 1, wherein the controller of the physiological monitoring system is configured to determine, based on the physiological signal data, the proper placement of the at least one sensor.

3. The medical system of claim 1, wherein the controller of the mobile device is configured to determine, based on the physiological signal data, the proper placement of the at least one sensor.

4. The medical system of claim 1, wherein the proper placement is a location determined to facilitate receiving a physiological signal having a corresponding quality metric that satisfies a quality criterion.

5. The medical system of claim 4, wherein the quality metric comprises a value of at least one of image noise, resolution, contrast, and a signal to noise ratio (SNR).

6. The medical system of claim 1, wherein the information about the proper placement of the at least one sensor comprises a representation of the patient's body and a representation of the at least one sensor, wherein the representation of the at least one sensor is displayed at a location relative to the representation of the patient's body that corresponds to the proper position.

7. The medical system of claim 6, the mobile device further comprising an optical imaging component configured to obtain an image of the patient, wherein the representation of the patient's body is generated from the image of the patient.

8. The medical system of claim 1, wherein the information about the proper placement of the at least one sensor comprises instructions for positioning the at least one sensor.

9. The medical system of claim 1, wherein the physiological monitoring system comprises an electrocardiograph system, the at least one sensor comprising at least one electrode.

10. The medical system of claim 1, wherein the physiological monitoring system comprises an acoustic imaging system, the at least one sensor comprising at least one ultrasound transducer.

11. The medical system of claim 1, wherein the controller is further configured to receive, via the GUI, user input indicating parameters for an imaging task.

12. One or more computer-readable media having computer-executable instructions embodied thereon that, when executed by at least one processor, cause the at least one processor to instantiate at least one program component, the at least one program component comprising:
a positioning component configured to:
receive, from a physiological monitoring system, physiological signal data corresponding to a physiological signal obtained by a sensor, at a first sensor location, during a sensing task;
determine, based on the physiological signal data, that a first value of a quality metric, associated with the obtained physiological signal, fails to satisfy a quality criterion;
predict that a second value of the quality metric, associated with a physiological signal obtained by the sensor at a second sensor location, will satisfy the quality criterion; and
provide a positioning graphical user interface (GUI) on a display device, the GUI including information to direct relocation of the sensor to the second sensor location.

13. The media of claim 12, the at least one program component further comprising a rendering component configured to:
receive, from the physiological monitoring system, physiological signal data; and
cause the display device to present a representation of the physiological signal data.

14. The media of claim 12, the sensor comprising an ultrasound transducer.

15. A method of facilitating physiological monitoring of a patient using a physiological monitoring system, the physiological monitoring system comprising a controller and at least one sensor operatively coupled to the controller, wherein the physiological monitoring system is configured to communicate with a mobile device having a display device, the method comprising:
receiving, at the mobile device and from the physiological monitoring system, physiological signal data corresponding to a physiological signal obtained by a sensor, at a first sensor location, during a sensing task;
determining, based on the physiological signal data, that a first value of a quality metric, associated with the obtained physiological signal sensing data, fails to satisfy a quality criterion;
predicting that a second value of the quality metric, associated with a physiological signal obtained by the sensor at a second sensor location, will satisfy the quality criterion; and
causing the display device to present information to direct relocation of the sensor to the second sensor location.

16. The method of claim 15, wherein the physiological signal data comprises physiological signal data, and wherein determining that the first value of a quality metric fails to satisfy a quality criterion comprises:
determining the first value of the quality metric; and
comparing the first value of the quality metric to a quality criterion, wherein the quality criterion comprises at least one of a range and a threshold.

17. The method of claim 16, wherein the quality metric comprises at least one of image noise, resolution, contrast, and a signal to noise ratio (SNR).

18. The method of claim 15, wherein the physiological monitoring system comprises an acoustic imaging system, the at least one sensor comprising at least one ultrasound transducer.

19. The method of claim 15, further comprising receiving, via a GUI, user input indicating parameters for an imaging task, and providing the parameters to the physiological monitoring system.

20. The method of claim 15, further comprising:
receiving, from the physiological monitoring system, physiological signal data; and
causing the display device to present a representation of the physiological signal data.
